(19) United States
(12) Patent Application Publication
(10) Pub. No.: US 2024/0395784 A1
(43) Pub. Date: Nov. 28, 2024

(54) ELECTRONIC DEVICE AND INTERCONNECTION STRUCTURE

(71) Applicant: Advanced Semiconductor Engineering, Inc., Kaohsiung (TW)

(72) Inventors: Kuei-Hao TSENG, Kaohsiung (TW); Kai Hung WANG, Kaohsiung (TW); Chih Lung LIN, Kaohsiung (TW)

(73) Assignee: Advanced Semiconductor Engineering, Inc., Kaohsiung (TW)

(21) Appl. No.: 18/202,243

(22) Filed: May 25, 2023

(51) Int. Cl. H01L 25/16; A61B 5/256; H01L 23/00; H01L 23/31

(52) U.S. Cl. CPC H01L 25/16; A61B 5/256; H01L 23/3121; H01L 24/45; H01L 24/48; A61B 2562/0209; A61B 2562/04; A61B 2562/164; H01L 23/145; H01L 23/15; H01L 2224/45124; H01L 2224/45139; H01L 2224/45144; H01L 2224/45147; H01L 2224/45164; H01L 2224/45169; H01L 2224/48091; H01L 2224/48195; H01L 2224/48225

(57) ABSTRACT

The present disclosure provides an electronic device. The electronic device includes a flexible carrier, an electronic component disposed over the flexible carrier, and a first flexible connection element configured to connect the flexible carrier and the electronic component. The first flexible connection element is configured to extend along a deformation direction of the electronic device. An interconnection structure is also provided.

### BACKGROUND

### 1. Technical Field

The present disclosure relates to an electronic device.

### 2. Description of the Related Art

Sensors or detectors may be integrated into a wearable device to obtain signals or pieces of information reflecting physical activity and/or health. Conventionally, signals or pieces of information are transmitted through copper layers or conductive printing ink. The copper layers or conductive printing ink might deteriorate (crack or break) when the wearable device is stretched or curved to conform to a surface of a user's body. Transmission problems may become severe, affecting detection quality and device reliability.

### SUMMARY

In some arrangements, an electronic device includes a flexible carrier, an electronic component disposed over the flexible carrier, and a first flexible connection element configured to connect the flexible carrier and the electronic component. The first flexible connection element is configured to extend along a deformation direction of the electronic device.

In some arrangements, an electronic device includes a flexible carrier having a first surface, a second surface opposite to the first surface, and a lateral surface extending between the first surface and the second surface. The electronic device also includes an electronic component disposed over the second surface of the flexible carrier and a first sensing element disposed over the lateral surface of the flexible carrier. The first sensing element includes a first conductive base and a first connector at least partially embedded in the first conductive base. The first conductive base has a first sensing area for collecting a biological signal and substantially coplanar with the first surface of the flexible carrier.

In some arrangements, an interconnection structure includes a base having a recessed portion, a first connector at least partially embedded in the base, and a second connector at least partially embedded in the base. The interconnection structure also includes a flexible connection element connecting the first connector and the second connector and is configured to extend along a deformation direction of the interconnection structure. The recessed portion is disposed between the first connector and the second connector and is configured to provide adjustment to a relative position between the first connector and the second connector.

### BRIEF DESCRIPTION OF THE DRAWINGS

Aspects of some arrangements of the present disclosure are best understood from the following detailed description when read with the accompanying figures. It is noted that various structures may not be drawn to scale, and dimensions of the various structures may be arbitrarily increased or reduced for clarity of discussion.

FIG. 1A is a cross-section of an electronic device in accordance with some arrangements of the present disclosure.

FIG. 1B is a perspective view of a part of an electronic device in accordance with some arrangements of the present disclosure.

FIG. 1C is a conceptual view showing a state in which a user wears a part of an electronic device in accordance with some arrangements of the present disclosure.

FIG. 2A is a cross-section of an electronic device in accordance with some arrangements of the present disclosure.

FIG. 2B is a cross-section of an electronic device in accordance with some arrangements of the present disclosure.

FIG. 3 is a cross-section of an electronic device in accordance with some arrangements of the present disclosure.

FIG. 4 is a cross-section of an electronic device in accordance with some arrangements of the present disclosure.

FIG. 5A is a perspective view of a part of an electronic device in accordance with some arrangements of the present disclosure.

FIG. 5B is a perspective view of a part of an electronic device in accordance with some arrangements of the present disclosure.

FIG. 5C is a perspective view of a part of an electronic device in accordance with some arrangements of the present disclosure.

FIG. 5D is a cross-section of a part of an electronic device in accordance with some arrangements of the present disclosure.

FIG. 5E is a top view of an electronic device in accordance with some arrangements of the present disclosure.

FIG. 6A, FIG. 6B, FIG. 6C, FIG. 6D, FIG. 6E, and FIG. 6F are cross-sections of one or more stages of a method of manufacturing an electronic device in accordance with an arrangement of the present disclosure.

FIG. 7A, FIG. 7B, and FIG. 7C are cross-sections of one or more stages of a method of manufacturing an electronic device in accordance with an arrangement of the present disclosure.

### DETAILED DESCRIPTION

The following disclosure provides for many different arrangements, or examples, for implementing different features of the provided subject matter. Specific examples of components and arrangements are described as follows to explain certain aspects of the present disclosure. These are, of course, merely examples and are not intended to be limiting. For example, the formation of a first feature over or on a second feature in the description that follows may include arrangements in which the first and second features are formed or disposed in direct contact, and may also include arrangements in which additional features may be formed or disposed between the first and second features, such that the first and second features may not be in direct contact. In addition, the present disclosure may repeat reference numerals and/or letters in the various examples. This repetition is for the purpose of simplicity and clarity and does not in itself dictate a relationship between the various arrangements and/or configurations discussed.

Spatial descriptions, such as “above,” “below,” “up,” “left,” “right,” “down,” “top,” “bottom,” “vertical,” “horizontal,” “side,” “higher,” “lower,” “upper,” “over,” “under,” and so forth, are indicated with respect to the orientation shown in the figures unless otherwise specified. It should be understood that the spatial descriptions used herein are for purposes of illustration only, and that practical implementations of the structures described herein can be spatially arranged in any orientation or manner, provided that the merits of the arrangements of this disclosure are not deviated from by such arrangement.

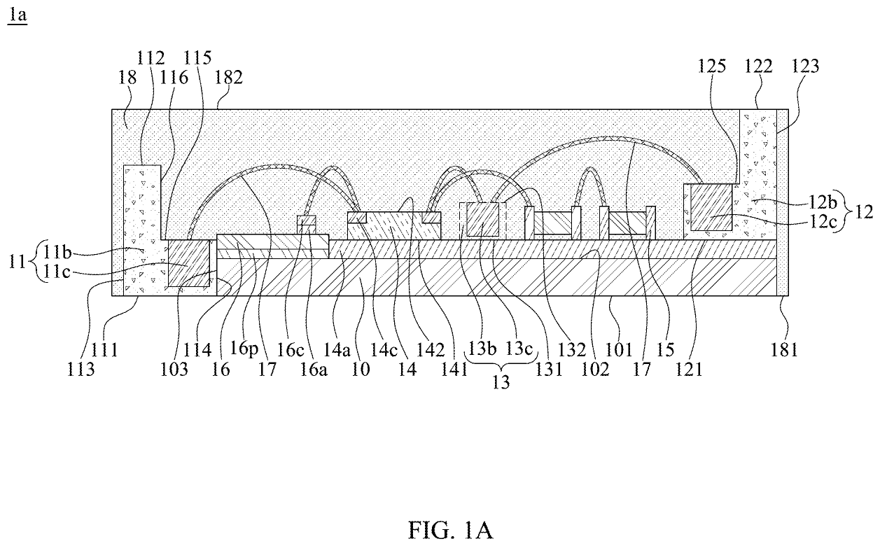

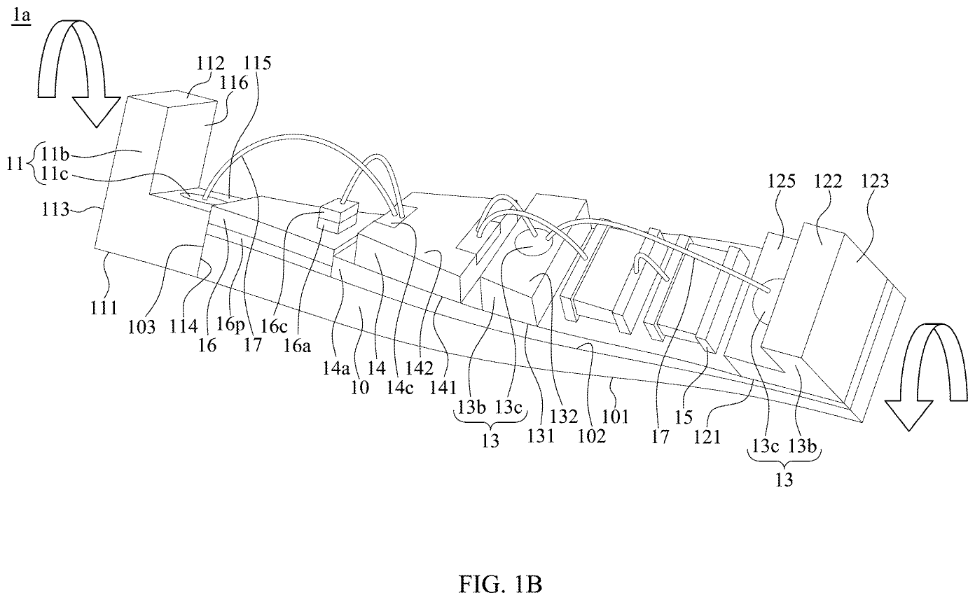

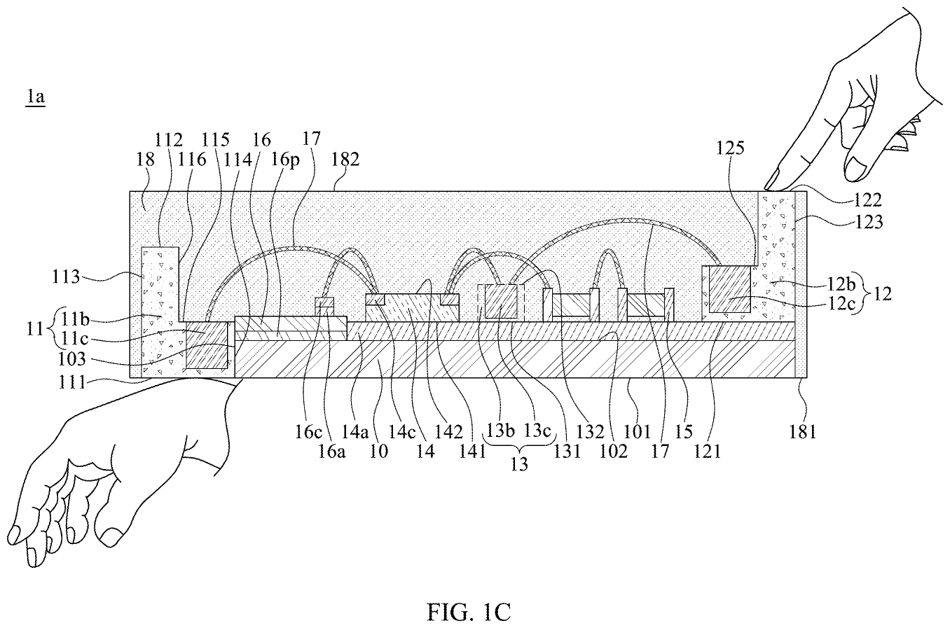

FIG. 1A is a cross-section of an electronic device 1a in accordance with some arrangements of the present disclosure. In some arrangements, the electronic device 1a may include or be a part of different types of wearable items, such as a smartwatch, a bracelet, a pair of glasses, goggles, a piece of jewelry, a piece of clothing, earphones, headphones, a helmet, or any type of wearable item that can be worn by and/or attached to an object or a target. The object may include a human or an animal. Configuration or application of the electronic device 1a described hereafter is for illustrative purposes only, and not intended to limit the present disclosure.

In some arrangements, the electronic device 1a may be a piece of equipment that detects or collects an external signal by using various detection devices (such as sensors). In some arrangements, the electronic device 1a may perform data communication with a base station or a terminal device (such as a mobile phone) in a wireless communications manner, such as via radio frequency identification technology or short-range wireless communications technology. In some arrangements, the electronic device 1a may be used in combination with a detection device (such as a sensor), an electronic device (such as a signal processing device) and/or other corresponding external devices for further processing acquired signals.

Referring to FIG. 1A, the electronic device 1a may include a carrier 10, sensing elements 11, 12, an interconnection structure 13, electronic components 14, 15, an antenna element 16, one or more flexible connection elements 17, and an encapsulant 18.

The carrier 10 may include a supporting element structurally supporting the sensing elements 11, 12, the interconnection structure 13, the electronic components 14, 15, and the antenna element 16. In some arrangements, the carrier 10 may include or be a part of a band, a strap, a loop, or a bracelet.

In some arrangements, the carrier 10 may be configured to maintain a relative distance (or a relative location) between the electronic device 1a and an object or a target to be detected. For example, when the electronic device 1a is worn by a user, the carrier 10 may be configured to make the sensing element 11 be adjacent (or close) to the user's skin. For example, the carrier 10 may be configured to prevent the sensing element 11 from sliding or otherwise being displaced on the user's skin.

In some arrangements, the carrier 10 may include a surface 101, a surface 102 opposite to the surface 101, and a surface (or lateral surface) 103 extending between the surface 101 and the surface 102. The surface 101 of the carrier 10 may be configured to face (or contact) an object or a target to be detected. For example, when the electronic device 1a is worn by a user, the surface 101 of the carrier 10 may face the user's skin.

In some arrangements, the carrier 10 may include a dielectric material. The dielectric material may include, for example, but is not limited to, phosphoric anhydride (PA), a polyimide (PI), a polybenzoxazole (PBO), Borophosphosilicate Glass (BPSG), Undoped Silicate Glass (USG), silicon oxide, silicon nitride, silicon oxynitride, any combination of two or more thereof, or the like. In some arrangements, the carrier 10 may include rubber, silicon, polyester, polyurethane, a liquid silicone rubber (LSR), fluoroelastomer (FKM), or other suitable materials such as an elastic material, a soft material, a sponge-like material, or a flexible material. In some arrangements, the carrier 10 may include a flexible carrier, an elastic carrier, an adjustable carrier, etc. In some arrangements, the carrier 10 may be configured to be adjustable. For example, when the electronic device 1a is worn by a user, the carrier 10 may flexibly adjust its shape to conform to a body part of the user. The carrier 10 may be soft and flexible enough for the user to wear comfortably for an extended time.

In some arrangements, the carrier 10 may be exclusive of an interconnection structure, such as a redistribution layer (RDL), a circuit layer, a conductive trace, a conductive via, etc. For example, there may be no interconnection structure in the carrier 10. For example, the signal transmissions may be conducted through the flexible connection elements 17 without passing through the carrier 10. For example, the signal transmissions may be conducted outside of the carrier 10.

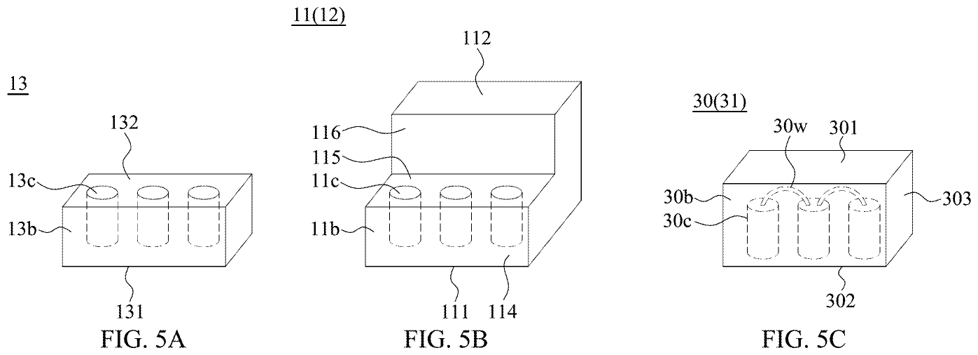

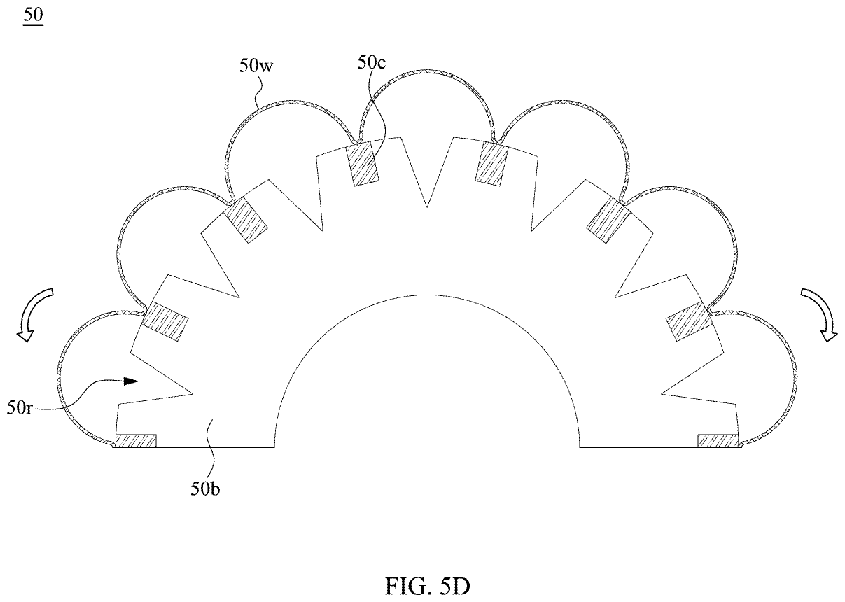

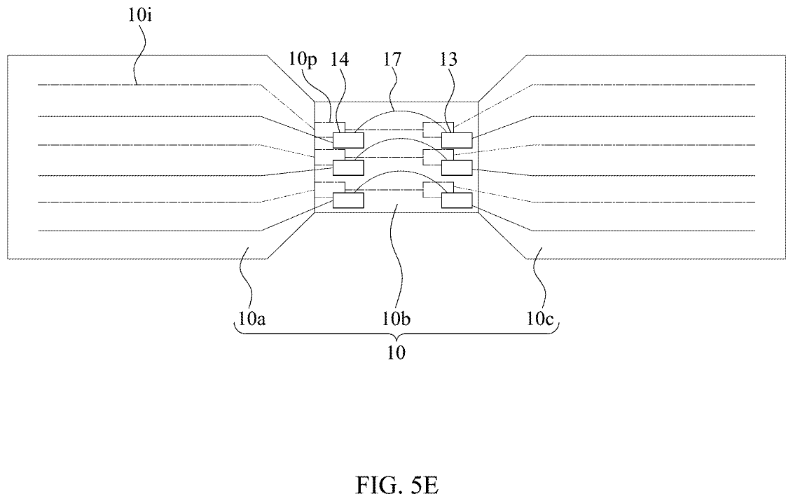

However, in some other arrangements, the carrier 10 may include an interconnection structure (such as the interconnection structure 10i in FIG. 5E) and may include one or more conductive pads (such as the conductive pad 10p in FIG. 5E) in proximity to, adjacent to, or embedded in and exposed by the surface 101 and/or 102 of the carrier 10. The interconnection structure in carrier 10 may be configured to support the signal transmissions in addition to the flexible connection elements 17.

The sensing element 11 may be disposed over the surface 103 of the carrier 10. The sensing element 11 may contact (such as directly contact) the surface 103 of the carrier 10. The sensing element 11 may be supported or fixed on the surface 103 of the carrier 10.

The sensing element 11 may include a surface 111, a surface 112 opposite to the surface 111, and a surface (or lateral surface) 113 extending between the surface 111 and the surface 112. The sensing element 11 may include a surface (or lateral surface) 114 opposite to the surface 113 and contacting the carrier 10, a surface 115 opposite to the surface 111, and a surface 116 extending between the surface 112 and the surface 115. The surface 116, the surface 115, and the surface 114 may define a stepped structure. The surface 116 and the surface 115 may define a recess. The surface 115 may be substantially parallel to the surface 112 and recessed from the surface 112. The surface 112 may protrude from the surface 115. The surface 116 may be parallel to the surface 114 and recessed from the surface 114. The surface 114 may protrude from the surface 116.

In some arrangements, the surface 111 of the sensing element 11 and the surface 101 of the carrier 10 may together form a surface facing (or contacting) an object or a target to be detected. In some arrangements, the surface 111 of the sensing element 11 and the surface 101 of the carrier 10 may be substantially coplanar. In some arrangements, the surface 111 of the sensing element 11 may include or be a part of a band, a strap, a loop, or a bracelet.

The sensing element 11 may include a base 11b and a connector 11c. In some arrangements, the base 11b may be conductive. For example, the base 11b may include electrically conductive polymers or electrically conductive polymer composites. For example, the base 11b may include conductive fillers with high conductivity and insulating polymer matrices. For example, the base 11b may include conductive LSR (CLSR), which may include conductive fillers, such as carbon blacks, carbon nanotubes, copper, silver, etc.

In some arrangements, similar to the carrier 10, the base 11b may be configured to be flexible or adjustable. In some arrangements, the carrier 10 and the base 11b may have different characteristics or properties. For example, the electrical conductivity of the base 11b may exceed the electrical conductivity of the material of the carrier 10. For example, the base 11b may be conductive and the material of the carrier 10 may be non-conductive.

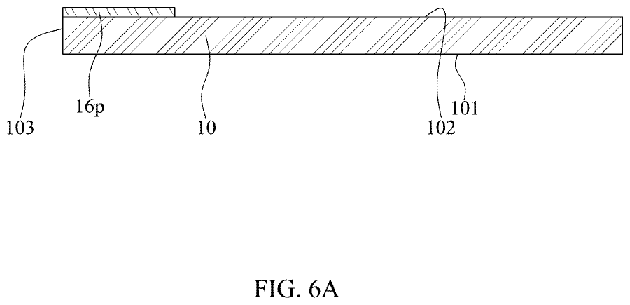

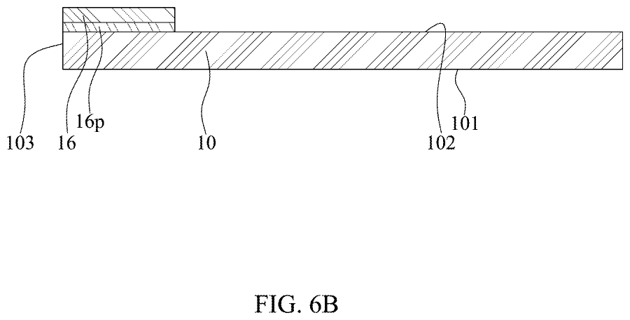

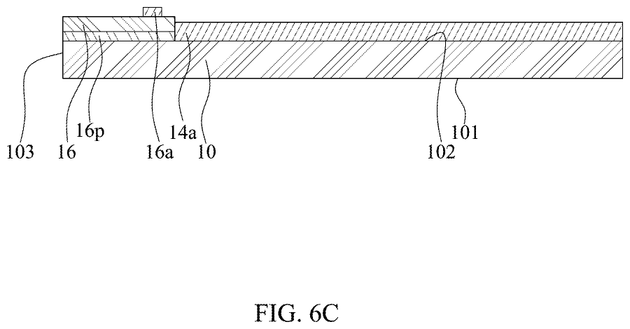

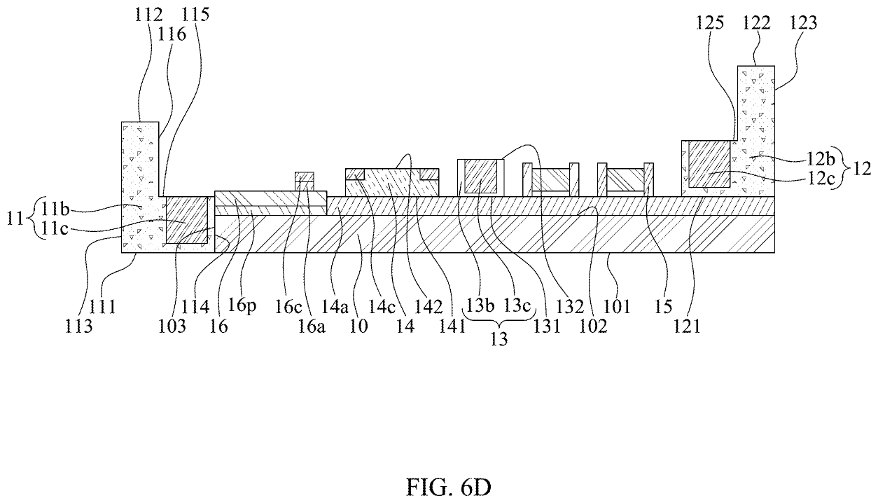

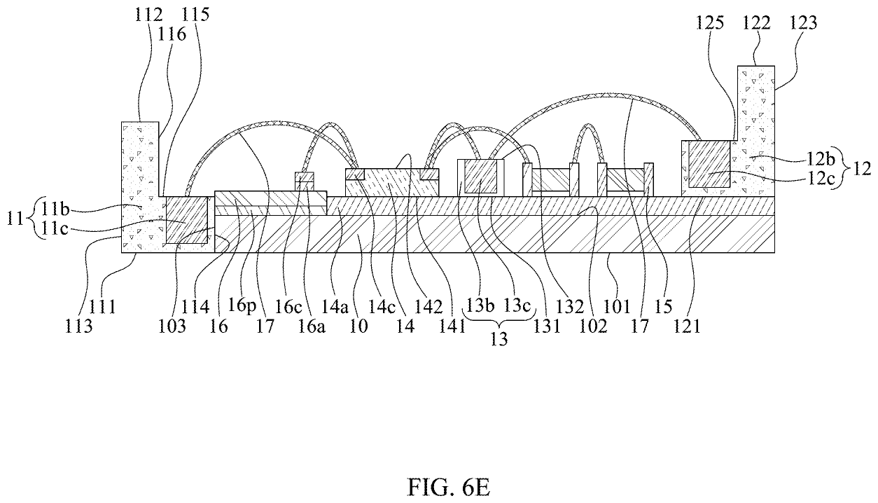

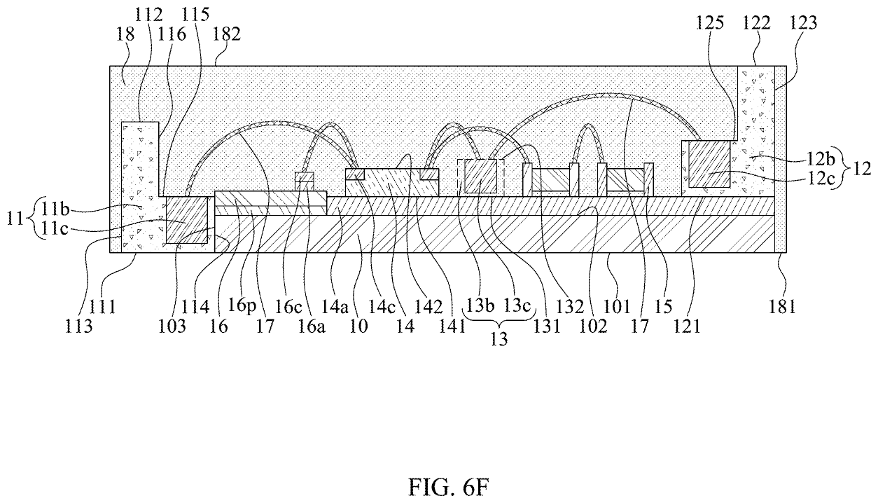

The connector 11c may be in proximity to, adjacent to, or embedded in and exposed by the surface 115 of the sensing element 11. The connector 11c may be at least partially exposed by the surface 115 of the sensing element 11. The connector 11c may not be exposed by the surface 111 of the sensing element 11. In some arrangements, the connector 11c may include a metal block, a conductive pad, a conductive pillar (e.g., a copper pillar), a conductive via, a conductive wire, a solder ball, etc. In some arrangements, the connector 11c may increase the structural stability during the bonding operation (shown in FIG. 6E); and thus, manufacturing yield can be increased.

The sensing element 12 may be disposed over the surface 102 of the carrier 10. The sensing element 12 may be connected to the surface 102 of the carrier 10 through an adhesive layer 14a. The adhesive layer 14a may include a die attach film (DAF), a glue, a bonding layer, an underfill, or another suitable material.

The sensing element 12 may include a surface 121, a surface 122 opposite to the surface 121, and a surface (or lateral surface) 123 extending between the surface 121 and the surface 122. The sensing element 12 may include a surface 125 opposite to the surface 121 and recessed from the surface 122.

The sensing element 12 may include a base 12b and a connector 12c. The connector 12c may be in proximity to, adjacent to, or embedded in and exposed by the surface 125 of the sensing element 11. The base 12b and the connector 12c may be similar to the base 11b and the connector 11c. Therefore, some details may correspond to the preceding description and are thus not repeated for brevity.

In some arrangements, the sensing elements 11 and 12 may be formed in the same process or by the same tool or mold to reduce the manufacturing cost of the electronic device 1a. Therefore, the sensing elements 11 and 12 may have the same structure.

The sensing elements 11 and 12 may each include an electrode, a thermistor, a pressure sensor, a proximity sensor, a motion sensor, an acoustic sensor, a smell sensor, a particle sensor, a humidity sensor, an optical transmitter, an optical receiver, an optical transceiver, or a combination thereof.

The sensing elements 11 and 12 may each be configured to detect signals (which may be single-ended signals or differential signals) pieces of information, or information packets, such as biological signals (or biosignals), physiological signals, motions (e.g., body motions of a human or animal), and/or environmental information in a vicinity of an object or a target. For example, when the electronic device 1a is worn by a user, the sensing elements 11 and 12 may be configured to detect or collect one or more signals or pieces of information associated with the user. For example, the sensing elements 11 and 12 may each be configured to detect light, sound, temperature, air pressure, smell, particle, humidity, or other environmental variables around the user.

In some arrangements, the surface 111 of the sensing element 11 and the surface 122 of the sensing element 12 may each include or define a sensing area for contacting (such as directly contact) an object or a target to be detected. For example, the surface 111 of the sensing element 11 and the surface 122 of the sensing element 12 may each be configured to detect or collect a biological signal. For example, as shown in FIG. 1C, when the electronic device 1a is worn on a portion (such as a first portion) of the user's skin, the surface 111 of the sensing element 11 may contact the portion (such as the first portion) of the user's skin, and the surface 122 of the sensing element 12 may be contacted by another portion (such as a second portion) of the user's skin. In some arrangements, the electronic device 1a is worn on the left wrist of a user, the surface 111 of the sensing element 11 may contact the skin of the left wrist of the user and the user may contact the surface 122 of the sensing element 12 with his or her right-hand finger.

In some arrangements, the sensing elements 11 and 12 may each be electrically connected to the electronic component 14 through the flexible connection elements 17. In some arrangements, the signals detected by the sensing elements 11 and 12 may be further processed by the electronic components 14 to determine a biological parameter of the user, such as a pulse travel time (PTT), an electroencephalogram (EEG), electrocardiogram (ECG), electromyogram (EMG), electrooculogram (EOG), galvanic skin response (GSR), sweat composition, pH, heart rate variability (HRV), or other biologically-relevant information associated with the user.

The interconnection structure 13 may be disposed over the surface 102 of the carrier 10. The interconnection structure 13 may be connected to the surface 102 of the carrier 10 through the adhesive layer 14a. The interconnection structure 13 may include a surface 131 and a surface 132 opposite to the surface 131. The surface 131 may face (or contact) the surface 102 of the carrier 10.

The interconnection structure 13 may include a base 13b and a connector 13c. In some arrangements, the base 13b may include rubber, silicon, polyester, polyurethane, an LSR, FKM, or other suitable materials such as an elastic material, a soft material, a sponge-like material, or a flexible material. In some arrangements, similar to the carrier 10, the base 13b may be configured to be flexible or adjustable.

In some arrangements, the base 13b may be conductive. In some arrangements, the base 13b and the base 11b may have the same characteristics or properties.

In some arrangements, the base 13b and the base 11b may have different characteristics or properties. For example, the electrical conductivity of the base 11b may exceed the electrical conductivity of the base 13b. For example, the base 11b may be conductive and the base 13b may be non-conductive.

The connector 13c may be in proximity to, adjacent to, or embedded in and exposed by the surface 132 of the interconnection structure 13. The connector 13c may be at least partially exposed by the surface 132 of the interconnection structure 13. The connector 13c may not be exposed by the surface 131 of the interconnection structure 13. In some arrangements, the connector 13c may include a metal block, a conductive pillar, a conductive via, a conductive wire, a solder ball, etc.

The electronic component 14 may be disposed over the surface 102 of the carrier 10. The electronic component 14 may be connected to the surface 102 of the carrier 10 through the adhesive layer 14a. The electronic component 14 and the sensing element 11 may not overlapped in a direction substantially perpendicular to the surface 102 of the carrier 10.

In some arrangements, the electronic component 14 may include a surface 141 facing the carrier 10 and a surface 142 opposite to the surface 141. The surface 141 may face (or contact) the surface 102 of the carrier 10. In some arrangements, the surface 142 may include an active surface and the surface 141 may include a backside or rear surface. In some arrangements, the electronic component 14 may include one or more conductive pads 14c in proximity to, adjacent to, or embedded in and exposed by the surface 142.

The electronic component 14 may include a processor, a controller, a memory, or an input/output (I/O) buffer, etc. For example, the electronic component 14 may be configured to process (e.g., analyze, modify, synthesize, convert to digital signal, amplify, etc.), to store, and/or transmit signals detected by the sensing elements 11 and 12. In some arrangements, the electronic component 14 may include, for example, a central processing unit (CPU), a microprocessor unit (MPU), a graphics processing unit (GPU), a microcontroller unit (MCU), an application-specific integrated circuit (ASIC), a field-programmable gate array (FPGA), or another type of integrated circuit.

The electronic component 15 may be disposed over the surface 102 of the carrier 10. The electronic component 15 may be connected to the surface 102 of the carrier 10 through the adhesive layer 14a.

The electronic component 15 may include a passive component requiring no external power source and providing no electrical gain. For example, the electronic component 15 may include a resistor, a capacitor, an inductor, a transformer, a diode, a thermistor, a varactor, a transducer, etc.

The antenna element 16 may be disposed over the surface 102 of the carrier 10. The antenna element 16 may be connected to the surface 102 of the carrier 10 through a primer layer 16p.

The primer layer 16p may act as an intermediary tie layer between the antenna element 16 and the carrier 10. The primer layer 16p may promote adhesion between the antenna element 16 and the carrier 10 and may include a single primer layer or a plurality of primer layers. The primer layer 16p may include at least one of one or more polyurethane polymers, one or more phenoxy polymers, and one or more polyvinyl chloride polymers. In some arrangements, the primer layer 16p and the antenna element 16 may be disposed over the adhesive layer 14a.

In some arrangements, the antenna element 16 may include a patch antenna, such as a planar inverted-F antenna (PIFA) or another feasible type. In some arrangements, the antenna element 16 may include conductive ink, a wire, a metal strip, a metal ribbon, multiple wires, multiple metal strips, multiple metal ribbons, a wire mesh, perforated metal, a metal coating, etc. In some arrangements, the antenna element 16 may include a conductive material such as metal or a metal alloy. Examples of the conductive material include gold (Au), silver (Ag), aluminum (Al), copper (Cu), platinum (Pt), Palladium (Pd), other metal(s) or alloy(s), or a combination thereof.

The antenna element 16 may include a connector 16c. In some arrangements, the connector 16c may include a metal block, a conductive pad, a conductive pillar, a conductive via, a conductive wire, a solder ball, etc. In some arrangements, the connector 16c may be connected to the antenna element 16 through an adhesive layer 16a. The adhesive layer 16a may include a conductive DAF, conductive glue, or other suitable material.

In some arrangements, the antenna element 16 may be electrically connected to the electronic component 14 through the flexible connection elements 17. In some arrangements, the signals processed by the electronic components 14 may be transmitted to the antenna element 16 through the flexible connection element 17, the connector 16c, and the adhesive layer 16a. In some arrangements, the signals processed by the electronic components 14 may be transmitted or radiated by the antenna element 16.

In some arrangements, the sensing elements 11, 12, the interconnection structure 13, the electronic components 14, 15, and the antenna element 16 may each be electrically connected, and the electrical connections may be attained by the flexible connection elements 17. For example, the flexible connection elements 17 may provide, constitute, or establish signal transmission paths among the sensing elements 11, 12, the interconnection structure 13, the electronic components 14, 15, and the antenna element 16.

The flexible connection elements 17 may include conductive wires. However, in some arrangements, the flexible connection elements 17 may include conductors of other shapes or configurations, such as wire bundles, cables, pillars, rods, rails, pipes, etc. In some arrangements, the flexible connection elements 17 may include conductive material such as metal or a metal alloy. Examples of the conductive material include gold (Au), silver (Ag), aluminum (Al), copper (Cu), platinum (Pt), Palladium (Pd), other metal(s) or alloy(s), or a combination of two or more thereof.

In some arrangements, the flexible connection elements 17 may be configured to receive signals from the sensing element 11 and transmit the signals to the electronic component 14. For example, one of the flexible connection elements 17 may include an end electrically connected to the connector 11c of the sensing element 11 and an opposite end electrically connected to the conductive pad 14c of the electronic component 14. The length of the flexible connection element 17 connecting the sensing element 11 and the electronic component 14 may be greater than (or exceed) the horizontal distance (e.g., the shortest horizontal distance) therebetween.

In some arrangements, the flexible connection elements 17 may be configured to receive processed signals from the electronic component 14 and transmit the processed signals to the antenna element 16. For example, one of the flexible connection elements 17 may include an end electrically connected to the conductive pad 14c of the electronic component 14 and an opposite end electrically connected to the connector 16c of the antenna element 16. The length of the flexible connection element 17 connecting the electronic component 14 and the antenna element 16 may be greater than the horizontal distance (e.g., the shortest horizontal distance) therebetween.

In some arrangements, the flexible connection elements 17 may be configured to receive signals from the sensing element 12 and transmit the signals to the interconnection structure 13. For example, one of the flexible connection elements 17 may include an end electrically connected to the connector 12c of the sensing element 12 and an opposite end electrically connected to the connector 13c of the interconnection structure 13. The length of the flexible connection element 17 connecting the sensing element 12 and the interconnection structure 13 may be greater than the horizontal distance (e.g., the shortest horizontal distance) therebetween.

The interconnection structure 13 may be configured to function as a relay point, a bridge, and intermediate station for connecting the flexible connection elements 17. For example, one of the flexible connection elements 17 may include an end electrically connected to the connector 13c of the interconnection structure 13 and an opposite end electrically connected to the conductive pad 14c of the electronic component 14. The length of the flexible connection element 17 connecting the interconnection structure 13 and the electronic component 14 may be greater than the horizontal distance (e.g., the shortest horizontal distance) therebetween. By using the interconnection structure 13, the flexible connection element 17 connecting the interconnection structure 13 and the electronic component 14 can be shorter and the device reliability can be increased.

The encapsulant 18 may be disposed over the surface 102 of the carrier 10. The encapsulant 18 may cover or encapsulate the sensing elements 11, 12, the interconnection structure 13, the electronic components 14, 15, the antenna element 16, and the flexible connection elements 17.

The surface 111 of the sensing element 11 and the surface 122 of the sensing element 12 may each be at least partially exposed by the encapsulant 18 to define a sensing area for contacting (such as directly contact) an object or a target to be detected. The surface 111 of the sensing element 11 and a surface 181 of the encapsulant 18 may be substantially coplanar. The surface 122 of the sensing element 12 and a surface 182 of the encapsulant 18 may be substantially coplanar. The connector 11c may be at least partially exposed by the base 11b and covered by the encapsulant 18.

In some arrangements, the encapsulant 18 may include rubber, silicon, polyester, polyurethane, an LSR, FKM, or other suitable materials such as an elastic material, a soft material, a sponge-like material, or a flexible material. In some arrangements, similar to the carrier 10, the encapsulant 18 may be configured to be flexible or adjustable.

In some arrangements, the encapsulant 18 and the base 11b may have different characteristics or properties. For example, the electrical conductivity of the base 11b may exceed the electrical conductivity of the encapsulant 18. For example, the base 11b may be conductive and the encapsulant 18 may be non-conductive.

In some arrangements, the encapsulant 18 and the base 13b may have the same material and the interface between the encapsulant 18 and the base 13b may not be observed. In such arrangements, the connector 13c of the interconnection structure 13 may float or be suspended in the encapsulant 18.

In some arrangements, two or more of the material of the carrier 10, the base 11b, the base 12b, the base 13b, and the encapsulant 18 may have similar or matched coefficients of thermal expansion (CTE). For example, two or more of the material of the carrier 10, the base 11b, the base 12b, the base 13b, and the encapsulant 18 may have similar or matched thermal expansion response to thermo-mechanical deformation. Thermo-mechanical deformation techniques may include, for example, hot-rolling, cold-rolling, plane strain compression, bi-axial tension, conform processing, bending, drawing, swaging, annealing, sintering, monotonic tension processing, monotonic compression processing, monotonic torsion processing, cyclic thermal training under stress, and combinations thereof.

FIG. 1B is a perspective view of a part of the electronic device 1a in accordance with some arrangements of the present disclosure. For simplicity and clarity, the encapsulant 18 in FIG. 1A is omitted from FIG. 1B.

As the arrows indicate, the electronic device 1a may be twisted or rotated. For example, a part of the electronic device 1a may be twisted in a clockwise direction and an opposite part of the electronic device 1a may be twisted in a counterclockwise direction. The electronic device 1a may be twisted such that two portions of the surface 101 of the carrier 10 may face two opposite directions.

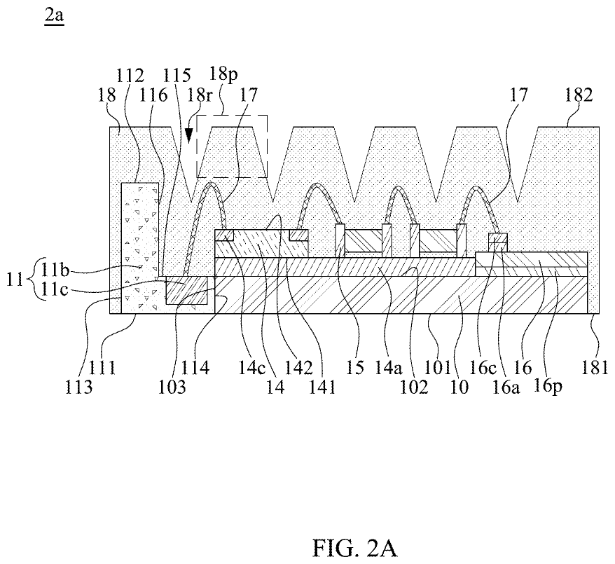

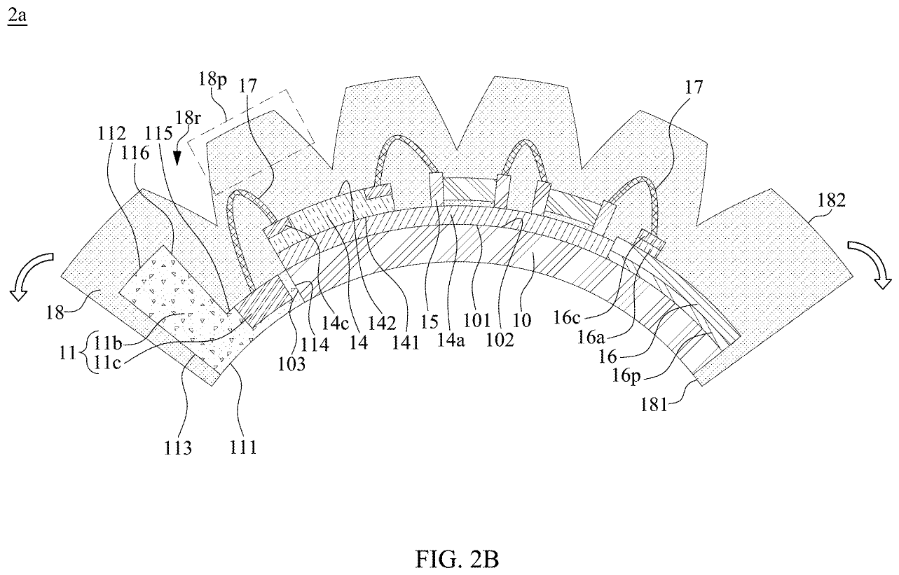

In some arrangements, the electronic device 1a may be stretched in two opposite directions. For example, the electronic device 1a may be curved into an arc (like the electronic device 2b shown in FIG. 2B). For example, two portions of the surface 102 of the carrier 10 may be stretched to face two opposite directions. For example, two portions of the surface 101 of the carrier 10 may face each other.

In a comparative arrangement, the signals or pieces of information detected by the sensors may be transmitted through copper layers or conductive printing ink. For examples, the electrical connections among the components (e.g., the sensing elements 11, 12, the interconnection structure 13, the electronic components 14, 15, and the antenna element 16) of the electronic device 1a may be attained by the carrier 10. The copper layers or conductive printing ink of the carrier 10 might deteriorate (crack or break) when the electronic device 1a is stretched or curved to conform to a surface of a user's body. Transmission problems may become severe, affecting detection quality and device reliability.

According to some arrangements of the present disclosure, the electrical connections among the components (e.g., the sensing elements 11, 12, the interconnection structure 13, the electronic components 14, 15, and the antenna element 16) of the electronic device 1a may be attained by the flexible connection elements 17.

The flexible connection elements 17 may extend along the deformation direction (or deformation orientation) of the electronic device 1a. For example, the flexible connection elements 17 may extend along the force direction of the electronic device 1a.

For example, the flexible connection elements 17 may extend along the twisting or rotating directions of the electronic device 1a shown in FIG. 1B. For example, the flexible connection elements 17 may extend along the stretching or bending directions of the electronic device 2a shown in FIG. 2B.

The flexible connection elements 17 may be configured to be adjustable. In comparison with the copper layers or conductive printing ink, the flexible connection elements 17 may be relatively more resistant to squeezing, stretching, or twisting, or other physical or structural changes. For example, the flexible connection elements 17 may be resilient, such that, after being squeezed, stretched, or twisted, it can return to its original state without deteriorating (such as cracking or breaking). Therefore, the electrical connections among the components of the electronic device 1a are stable. The components can maintain function while the electronic device 1a is being twisted, rotated, stretched, curved, pressed, pulled, deformed, etc. The detection efficiency of the electronic device 1a can be enhanced and the device reliability can be increased.

In addition, the locations of the components (e.g., the sensing elements 11, 12, the interconnection structure 13, the electronic components 14, 15, and the antenna element 16) of the electronic device 1a may not be limited by the routings of the carrier 10 and the design flexibility can be increased. Performance factors such as impedance interference, impedance matching, line widths, etc. can be considered and adjusted more easily.

Furthermore, since there may be no or less interconnection structure in the carrier 10, the carrier 10 can be thinner, the manufacturing cost of the electronic device 1a may be reduced and the electronic device 1a can be more compact and lightweight.

FIG. 2A is a cross-section of an electronic device 2a in accordance with some arrangements of the present disclosure. The electronic device 2a in FIG. 2A is similar to the electronic device 1a in FIG. 1A with differences therebetween as follows.

The sensing element 11, the electronic components 14 and 15, and the antenna element 16 may be arranged along the signal transmission direction of the electronic device 2a sequentially. For example, the sensing element 11 may detect or collect signals, and the signals may be transmitted to the electronic component 14 through the flexible connection elements 17. The electronic component 14 may process the signals, and the processed signals may be transmitted to the electronic components 15 and the antenna element 16 through the flexible connection elements 17. For example, the sensing element 11, the electronic components 14 and 15, and the antenna element 16 may be arranged according to the functions thereof sequentially to reduce the signal transmission path of the electronic device 2a and to avoid unnecessary wire crossing.

The flexible connection elements 17 may be arranged along the signal transmission direction of the electronic device 2a sequentially. For example, the flexible connection elements 17 may not overlap or cross from the cross-section of FIG. 2A. In addition, arranging the sensing element 11, the electronic components 14 and 15, and the antenna element 16 sequentially may facilitate the bonding operation for manufacturing the flexible connection elements 17; and thus, the manufacturing yield can be increased.

The location and number of the sensing element 11, the electronic components 14 and 15, and the antenna element 16 in FIG. 2A are for illustrative purposes only, and not intended to limit the present disclosure. For example, the electronic components 15 may be connected between the electronic component 14 and the antenna element 16. In some other arrangements, the electronic components 15 may be omitted. In some other arrangements, another sensing element (such as the sensing element 12 in FIG. 1A) may be disposed next to the antenna element 16 or next to the sensing element 11.

The encapsulant 18 may include recessed portions (or recessed regions) 18r recessed from the surface (or the top surface) 182 of the encapsulant 18 facing away from the carrier 10. The recessed portions 18r may define thinned portions of the encapsulant 18 that have smaller dimensions (such as thicknesses, widths, and/or cross sections) with respect to protruding portions 18p of the encapsulant 18. The protruding portions 18p may be separated by the recessed portions 18r. The protruding portions 18p and the recessed portions 18r may be alternated.

In some arrangements, the protruding portions 18p and the curved portions of the flexible connection elements 17 may be overlapped in a direction substantially perpendicular to the surface 102 of the carrier 10. For example, the protruding portions 18p and the apexes or the peaks of the connection elements 17 may be overlapped in a direction substantially perpendicular to the surface 102 of the carrier 10. For example, the two opposite ends of the connection elements 17 may not overlap the protruding portions 18p in a direction substantially perpendicular to the surface 102 of the carrier 10. For example, the ups and downs of the connection elements 17 may be corresponding to the protruding portions 18p and the recessed portions 18r.

FIG. 2B is a cross-section of the electronic device 2a in accordance with some arrangements of the present disclosure.

The sensing element 11, the electronic components 14 and 15, and the antenna element 16 may be arranged along the deformation direction of the electronic device 2a sequentially. The flexible connection elements 17 may be arranged along the deformation direction of the electronic device 2a sequentially. The flexible connection elements 17 may extend along the deformation direction of the electronic device 2a.

As the arrows indicate, the electronic device 2a may be stretched in two opposite directions. For example, the electronic device 2a may be curved into an arc. For example, two portions of the surface 102 of the carrier 10 may be stretched to face two opposite directions. For example, two portions of the surface 101 of the carrier 10 may face each other. The electronic device 2a may be curved, and the curved angle may be 90°, 180°, 270°, 360° or more.

According to some arrangements of the present disclosure, the recessed portions 18r of the electronic device 2a increase flexibility, such as increasing motion directions and curved angles. The recessed portions 18r may be configured to provide adjustment to a relative position between two of the protruding portions 18p.

For example, the electronic device 2a can accommodate curving in any direction, and may be twisted as shown in FIG. 1B. In some arrangements, the electronic device 2a may be relatively resistant to squeezing, stretching, or twisting, or other physical or structural changes. For example, the electronic device 2a may be resilient, such that, after being squeezed, stretched, or twisted, it can return to its original state without deteriorating (such as cracking or breaking). Therefore, the electrical connections among the components of the electronic device 2a are stable. The components can maintain function while the electronic device 2a is twisted, rotated, stretched, curved, pressed, pulled, deformed, etc. The detection efficiency of the electronic device 2a can be enhanced and the device reliability be increased.

In addition, when the electronic device 2a is worn by a user, the electronic device 2a can flexibly adjust its shape to conform to a body part of the user. Therefore, the position of the sensing element 11 relative the body part of the user can be adjusted accordingly. The sensing element 11 can adhere to the body part of the user. The sensibility of the sensing element 11 can be enhanced and the detection efficiency can be increased.

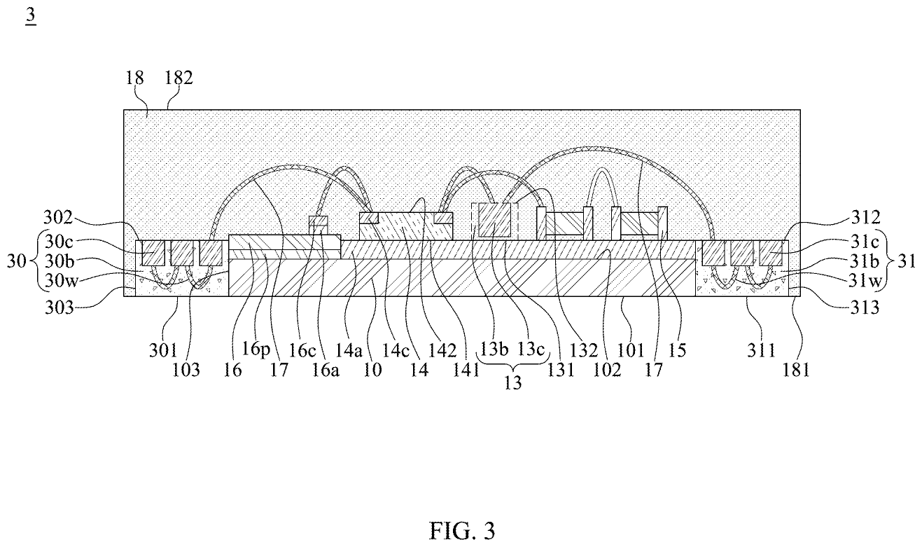

FIG. 3 is a cross-section of an electronic device 3 in accordance with some arrangements of the present disclosure. The electronic device 3 in FIG. 3 is similar to the electronic device 1a of FIG. 1A, differing in that the sensing elements 11 and 12 in FIG. 1A are replaced by sensing elements 30 and 31.

The sensing element 30 may be disposed over the surface 103 of the carrier 10. The sensing element 30 may contact (such as directly contact) the surface 103 of the carrier 10. The sensing element 30 may be supported or fixed on the surface 103 of the carrier 10.

The sensing element 30 may include a surface 301, a surface 302 opposite to the surface 301, and a surface (or lateral surface) 303 extending between the surface 301 and the surface 302.

The sensing element 30 may include a base 30b, a connector 30c, and a flexible connection element 30w. The base 30b and the connector 30c may be similar to the base 11b and the connector 11c in FIG. 1A. Therefore, some details may correspond to the preceding description and are thus not repeated for brevity.

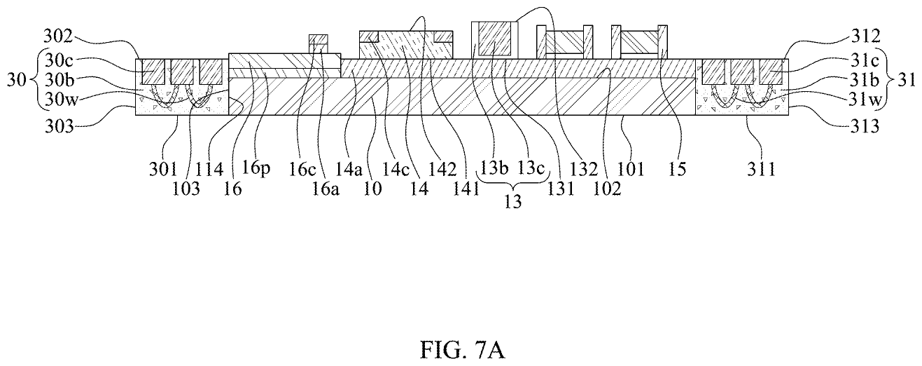

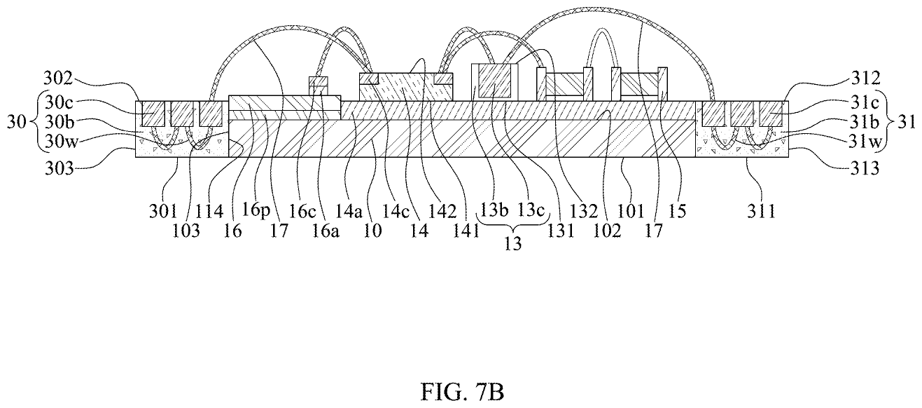

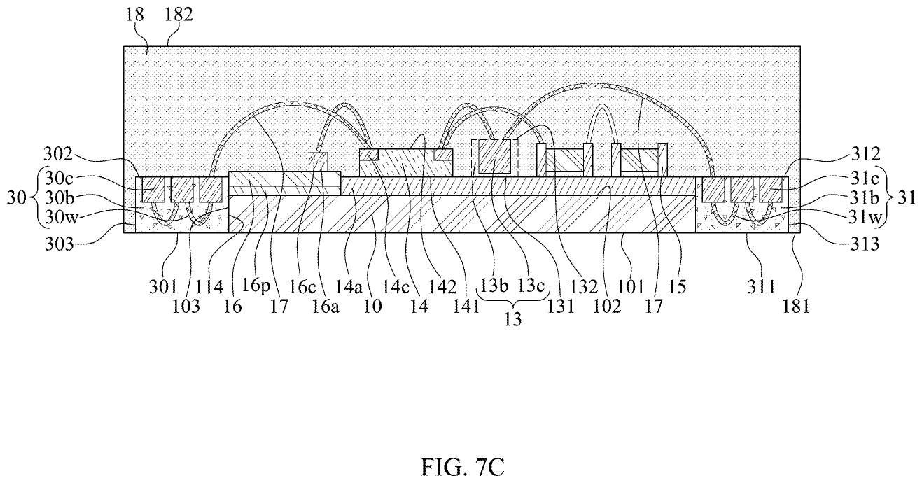

The connector 30c may be in proximity to, adjacent to, or embedded in and exposed by the surface 302 of the sensing element 30. The connector 30c may be at least partially exposed by the surface 302 of the sensing element 30. The connector 30c may not be exposed by the surface 301 of the sensing element 30. In some arrangements, the connector 30c may include a metal block, a conductive pad, a conductive pillar, a conductive via, a conductive wire, a solder ball, etc. In some arrangements, the connector 30c may increase the structural stability during the bonding operation (shown in FIG. 7B); and thus, the manufacturing yield can be increased.

The flexible connection elements 30w may be covered or encapsulated in the base 30b. The flexible connection elements 30w may include conductive wires. The flexible connection elements 30w may be similar to the flexible connection elements 17 in FIG. 1A. Therefore, some details may correspond to the preceding description and are thus not repeated for brevity.

The flexible connection elements 30w may each include an end electrically connected to one of the connectors 30c and an opposite end electrically connected to another one of the connectors 30c. The connectors 30c may each be configured to function as a relay point, a bridge, and an intermediate station for connecting the flexible connection elements 30w.

The flexible connection elements 30w may have a relatively lower impedance than the base 30b. For example, the flexible connection elements 30w may be configured to enhance, accelerate, or facilitate the signal transmission between the sensing area (e.g., the surface 301) of the base 30b and at least one of the connectors 30c. For example, the flexible connection elements 30w may be configured to increase the electrical conductivity between the sensing area (e.g., the surface 301) of the base 30b and at least one of the connectors 30c. For example, the flexible connection elements 30w may be configured to increase the signal strength between the sensing area (e.g., the surface 301) of the base 30b and at least one of the connectors 30c. Therefore, the signal noise can be reduced, the problem of transmission loss can be solved, and the detection quality can be improved.

The sensing element 31 may be disposed over a lateral surface of the carrier 10 opposite to the sensing element 30. The sensing element 31 may contact (such as directly contact) a lateral surface of the carrier 10 opposite to the sensing element 30. The sensing element 30 may be supported or fixed on a lateral surface of the carrier 10 opposite to the sensing element 30.

The sensing element 31 may include a surface 311, a surface 312 opposite to the surface 311, and a surface (or lateral surface) 313 extending between the surface 311 and the surface 312.

The sensing element 31 may include a base 31b, a connector 31c, and a flexible connection element 31w. The base 31b, the connector 31c, and the flexible connection element 31w may be similar to the base 30b, the connector 30c, and the flexible connection element 30w. Therefore, some details may correspond to the preceding description and are thus not repeated for brevity.

In some arrangements, the sensing elements 30 and 31 may be formed in the same process or by the same tool or mold to reduce the manufacturing cost of the electronic device 3. Therefore, the sensing elements 30 and 31 may have the same structure.

In some arrangements, the surface 301 of the sensing element 30 and the surface 311 of the sensing element 31 may each include or define a sensing area for contacting (such as directly contact) an object or a target to be detected.

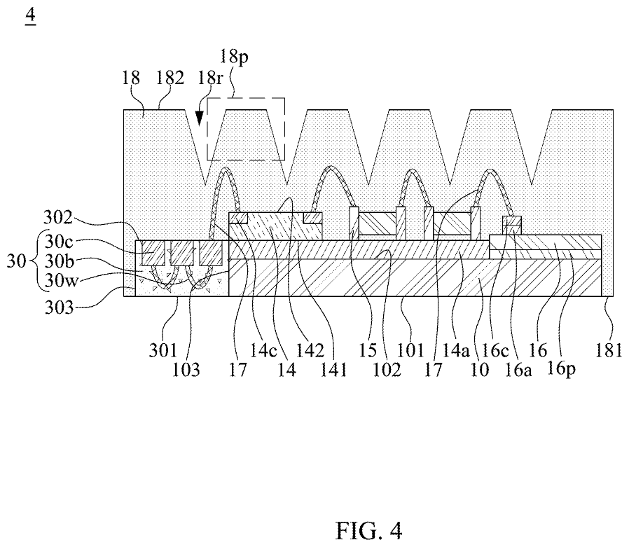

FIG. 4 is a cross-section of an electronic device 4 in accordance with some arrangements of the present disclosure. The electronic device 4 in FIG. 4 is similar to the electronic device 3 in FIG. 3 with differences therebetween as follows.

The sensing element 30, the electronic components 14 and 15, and the antenna element 16 may be arranged along the signal transmission direction of the electronic device 4 sequentially. For example, the sensing element 30 may detect or collect signals, and the signals may be transmitted to the electronic component 14 through the flexible connection elements 17. The electronic component 14 may process the signals, and the processed signals may be transmitted to the electronic components 15 and the antenna element 16 through the flexible connection elements 17. For example, the sensing element 30, the electronic components 14 and 15, and the antenna element 16 may be arranged according to the functions thereof sequentially to reduce the signal transmission path of the electronic device 4.

The flexible connection elements 17 may be arranged along the signal transmission direction of the electronic device 4 sequentially. For example, the flexible connection elements 17 may not overlap or cross one another from the cross-section of FIG. 4.

The location and number of the sensing element 30, the electronic components 14 and 15, and the antenna element 16 in FIG. 2A are for illustrative purposes only, and not intended to limit the present disclosure. For example, the electronic components 15 may be connected between the electronic component 14 and the antenna element 16. In some other arrangements, the electronic components 15 may be omitted. In some other arrangements, another sensing element (such as the sensing element 31 in FIG. 3) may be disposed next to the antenna element 16 or next to the sensing element 30.

Similar to the electronic device 2a in FIG. 2A, the encapsulant 18 may include recessed portions (or recessed regions) 18r which are recessed from the surface (or the top surface) 182 of the encapsulant 18 facing away from the carrier 10. The recessed portions 18r may define thinned portions of the encapsulant 18 that have smaller dimensions (such as thicknesses, widths, and/or cross sections) with respect to the protruding portions 18p of the encapsulant 18. Some details may correspond to the paragraphs with respect to FIG. 2A above, and description thereof is not repeated hereinafter for conciseness.

FIG. 5A is a perspective view of the interconnection structure 13 in accordance with some arrangements of the present disclosure. The interconnection structure 13 may include three connectors 13c in proximity to, adjacent to, or embedded in and exposed by the surface 132 of the interconnection structure 13. The location and number of the connectors 13c are for illustrative purposes only, and not intended to limit the present disclosure.

FIG. 5B is a perspective view of the sensing element 11 (or the sensing element 12) in accordance with some arrangements of the present disclosure. The sensing element 11 may include three connectors 11c in proximity to, adjacent to, or embedded in and exposed by the surface 115 of the sensing element 11. The location and number of the connectors 11c are for illustrative purposes only, and not intended to limit the present disclosure.

FIG. 5C is a perspective view of the sensing element 30 (or the sensing element 31) in accordance with some arrangements of the present disclosure. The sensing element 30 may include three connectors 30c in proximity to, adjacent to, or embedded in and exposed by the surface 302 of the sensing element 30. The location and number of the connectors 30c are for illustrative purposes only, and not intended to limit the present disclosure.

FIG. 5D is a cross-section of an interconnection structure 50 in accordance with some arrangements of the present disclosure.

The interconnection structure 50 may include a base 50b, a connector 50c, and a flexible connection element 50w. The interconnection structure 50 may be similar to the interconnection structure 13 of FIG. 1A and FIG. 3, differing in that the interconnection structure 50 includes the flexible connection elements 50w connecting the connectors 50c and the base 50b includes recessed portions 50r. In some arrangements, the interconnection structure 13 in FIG. 1A and FIG. 3 may be replaced with the interconnection structure 50. Detailed descriptions of the base 50b and the connector 50c may refer to the base 13b and the connector 13c with respect to FIG. 1A and FIG. 3.

The flexible connection elements 50w may be exposed by the base 50b. The flexible connection elements 50w may not be covered or encapsulated in the base 50b. The flexible connection elements 50w may include conductive wires.

The flexible connection elements 50w may include separated segments, each having opposite ends connecting to two of the connectors 50c. In some arrangements, the flexible connection elements 50w may be a single conductive wire. For example, the ends of the flexible connection elements 50w may be connected to each other. The connectors 50c may each be configured to function as a relay point, a bridge, and an intermediate station for connecting the flexible connection elements 50w.

Detailed descriptions of the flexible connection elements 50w may refer to the flexible connection elements 17 described with respect to FIG. 1A and FIG. 3.

In some arrangements, the sensing elements 11, 12, 30, and 31 may be replaced with the interconnection structure 50 provided that the base 50b is conductive. For example, the base 50b may include conductive fillers with high conductivity and insulating polymer matrices. For example, the base 50b may include conductive LSR (CLSR), which may include conductive fillers, such as carbon blacks, carbon nanotubes, copper, silver, etc.

As the arrows indicate, the interconnection structure 50 may be stretched in opposite directions. For example, the interconnection structure 50 may be curved into an arc. The interconnection structure 50 may be curved 90°, 180°, 270°, or 360°. According to some arrangements of the present disclosure, by forming the recessed portions 50r, the flexibility of the interconnection structure 50 may be increased, such as increasing motion directions and curved angles. The recessed portions 50r may be configured to provide adjustment to a relative position between two of the connectors 50c.

FIG. 5E is a top view of an electronic device in accordance with some arrangements of the present disclosure. In some arrangements, the electronic device in FIG. 5E may include or be a part of different types of wearable items, such as a pair of glasses, goggles, or any type of wearable item that can be worn by and/or attached to an object or a target. The electronic device in FIG. 5E may be similar to the electronic device 1a in FIG. 1A, with differences therebetween as follows.

In some arrangements, the carrier 10 may include portions 10a, 10b, and 10c. The portion 10a may be connected with the portion 10b. The portion 10a may be curved with respect to the portion 10b. The portion 10c may be connected with the portion 10b. The portion 10c may be curved with respect to the portion 10b.

In some arrangements, the carrier 10 may include an interconnection structure 10i, such as an RDL, a circuit layer, a conductive trace, a conductive via, etc. The interconnection structure 10i may be disposed within the carrier 10. The components (such as the electronic component 14 and the interconnection structure 13) over the carrier 10 may be connected through the flexible connection elements 17. The flexible connection elements 17 may be disposed outside of the carrier 10.

In some arrangements, the components (such as the electronic component 14 and the interconnection structure 13) over the carrier 10 may be electrically connected, and the electrical connections may be attained by the flexible connection elements 17 and the interconnection structure 10i.

By using the flexible connection elements 17, the locations of the components may not be limited by the routings of the carrier 10 and the design flexibility can be increased. Performance factors such as impedance interference, impedance matching, line widths, etc. can be considered and adjusted more easily. Furthermore, since there may be less interconnection structure 10i in the carrier 10, the carrier 10 are thinner, the manufacturing cost may be reduced and the electronic device can be more compact and lightweight.

For simplicity and clarity, the sensing elements 11, 12, the electronic components 15, the antenna element 16, and the encapsulant 18 in FIG. 1A are omitted from FIG. 5E. In some arrangements, the electronic device in FIG. 5E may include other components as shown in FIG. 1A according to design requirements.

FIG. 6A, FIG. 6B, FIG. 6C, FIG. 6D, FIG. 6E, and FIG. 6F illustrate cross-sections of one or more stages of a method of manufacturing an electronic device in accordance with an arrangement of the present disclosure. At least some of these figures have been simplified to better understand the aspects of the present disclosure. In some arrangements, the electronic device 1a may be manufactured through the operations described with respect to FIG. 6A, FIG. 6B, FIG. 6C, FIG. 6D, and FIG. 6F.

Referring to FIG. 6A, the primer layer 16p may be disposed over or on the carrier 10. In some arrangements, the primer layer 16p may be formed by printing, coating, or other suitable process. In some arrangements, the primer layer 16p may be cured.

Referring to FIG. 6B, the antenna element 16 may be disposed over or on the primer layer 16p. In some arrangements, the antenna element 16 may include a conductive ink and may be formed by printing, coating, or other suitable process. In some arrangements, the antenna element 16 may be cured.

Referring to FIG. 6C, the adhesive layer 16a may be disposed over or on the antenna element 16 and the adhesive layer 14a may be disposed over or on the carrier 10. In some arrangements, the adhesive layer 16a and the adhesive layer 14a may have different characteristics or properties. For example, the electrical conductivity of the adhesive layer 16a may exceed the electrical conductivity of the adhesive layer 14a. For example, the adhesive layer 16a may be conductive and the adhesive layer 14a may be non-conductive.

Referring to FIG. 6D, the sensing element 11 may be disposed over or on the surface 103 of the carrier 10. The sensing element 12, the interconnection structure 13, the electronic component 14, and the electronic component 15 are disposed over or on the surface 102 of the carrier 10.

Referring to FIG. 6E, the flexible connection elements 17 may be formed to connect the sensing element 11, the sensing element 12, the interconnection structure 13, the electronic component 14, the electronic component 15, and the antenna element 16. In some arrangements, the flexible connection elements 17 may be formed by wire bonding processes.

Referring to FIG. 6F, the encapsulant 18 may be disposed over the surface 102 of the carrier 10. In some arrangements, the encapsulant 18 may be formed by molding, such as by printing, compressive molding, transfer molding, liquid encapsulant molding, vacuum lamination, spin coating, or other suitable process.

A planarization operation or a grinding operation may be performed to remove a portion of the encapsulant 18 to expose the surface 111 of the sensing element 11 and/or the surface 122 of the sensing element 12. The planarization operation or grinding operation may include an abrasive machining process that uses a grinding wheel or grinder, a chemical mechanical planarization (CMP) process, an etching process, or a laser direct ablation (LDA) process.

A singulation may be performed to separate out individual package devices. The singulation may be performed, for example, by using a dicing saw, laser or other appropriate cutting techniques.

FIG. 7A, FIG. 7B, and FIG. 7C are cross-sections of one or more stages of a method of manufacturing an electronic device in accordance with an arrangement of the present disclosure. At least some of these figures have been simplified to better present aspects of the present disclosure. In some arrangements, the electronic device 3 may be manufactured through the operations described with respect to FIG. 6A, FIG. 6B, FIG. 6C, FIG. 7A, FIG. 7B, and FIG. 7C.

The operation in FIG. 7A may be subsequent to the operation in FIG. 6C. Referring to FIG. 7A, the sensing element 30 may be disposed over or on the surface 103 of the carrier 10. The sensing element 31 may be disposed over or on a lateral surface of the carrier 10 opposite to the sensing element 30. The interconnection structure 13, the electronic component 14, and the electronic component 15 may be disposed over or on the surface 102 of the carrier 10.

Referring to FIG. 7B, the flexible connection elements 17 may be formed to connect the sensing element 30, the sensing element 31, the interconnection structure 13, the electronic component 14, the electronic component 15, and the antenna element 16. In some arrangements, the flexible connection elements 17 may be formed by wire bonding processes.

Referring to FIG. 7C, the encapsulant 18 may be disposed over the surface 102 of the carrier 10. In some arrangements, the encapsulant 18 may be formed by molding, such as by printing, compressive molding, transfer molding, liquid encapsulant molding, vacuum lamination, spin coating, or other suitable process.

A planarization operation or a grinding operation may be performed to remove a portion of the encapsulant 18 to expose the surface 301 of the sensing element 30 and/or the surface 311 of the sensing element 31. Singulation may be performed to separate out individual package devices.

As used herein, the singular terms “a,” “an,” and “the” may include a plurality of referents unless the context clearly dictates otherwise.

As used herein, the terms “conductive,” “electrically conductive” and “electrical conductivity” refer to an ability to transport an electric current. Electrically conductive materials typically indicate those materials that exhibit little or no opposition to the flow of an electric current. One measure of electrical conductivity is Siemens per meter (S/m). Typically, an electrically conductive material is one having a conductivity greater than approximately 10⁴ S/m, such as at least 10⁵ S/m or at least 10⁶ S/m. The electrical conductivity of a material can sometimes vary with temperature. Unless otherwise specified, the electrical conductivity of a material is measured at room temperature.

As used herein, the terms “approximately,” “substantially,” “substantial” and “about” are used to describe and account for small variations. When used in conjunction with an event or circumstance, the terms can refer to instances in which the event or circumstance occurs precisely as well as instances in which the event or circumstance occurs to a close approximation. For example, when used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. For example, two numerical values can be deemed to be “substantially” the same or equal if a difference between the values is less than or equal to ±10% of an average of the values, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. For example, “substantially” parallel can refer to a range of angular variation relative to 0° that is less than or equal to ±10°, such as less than or equal to ±5°, less than or equal to ±4°, less than or equal to ±3°, less than or equal to ±2°, less than or equal to ±1°, less than or equal to ±0.5°, less than or equal to ±0.1°, or less than or equal to ±0.05°. For example, “substantially” perpendicular can refer to a range of angular variation relative to 90° that is less than or equal to ±10°, such as less than or equal to ±5°, less than or equal to ±4°, less than or equal to ±3°, less than or equal to ±2°, less than or equal to ±1°, less than or equal to ±0.5°, less than or equal to ±0.1°, or less than or equal to ±0.05°.

Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified.

While the present disclosure has been described and illustrated with reference to specific arrangements thereof, these descriptions and illustrations do not limit the present disclosure. It should be understood by those skilled in the art that various changes may be made and equivalents may be substituted without departing from the true spirit and scope of the present disclosure as defined by the appended claims. The illustrations may not be necessarily drawn to scale. There may be distinctions between the artistic renditions in the present disclosure and the actual apparatus due to manufacturing processes and tolerances. There may be other arrangements of the present disclosure which are not specifically illustrated. The specification and drawings are to be regarded as illustrative rather than restrictive. Modifications may be made to adapt a particular situation, material, composition of matter, method, or process to the objective, spirit and scope of the present disclosure. All such modifications are intended to be within the scope of the claims appended hereto. While the methods disclosed herein have been described with reference to particular operations performed in a particular order, it will be understood that these operations may be combined, sub-divided, or re-ordered to form an equivalent method without departing from the teachings of the present disclosure. Accordingly, unless specifically indicated herein, the order and grouping of the operations are not limitations of the present disclosure.

What is claimed is:

1. An electronic device, comprising:
a flexible carrier;
an electronic component disposed over the flexible carrier; and
a first flexible connection element configured to connect the flexible carrier and the electronic component,
wherein the first flexible connection element is configured to extend along a deformation direction of the electronic device.

2. The electronic device of claim 1, wherein the flexible carrier comprises a first conductive portion having a first sensing area for collecting a biological signal, and a non-conductive portion for supporting the electronic component.

3. The electronic device of claim 2, wherein the flexible carrier further comprises a first connector at least partially exposed from a surface of the first conductive portion opposite to the first sensing area of the first conductive portion.

4. The electronic device of claim 3, wherein the first flexible connection element has an end connecting to the first connector and an opposite end connecting to the electronic component.

5. The electronic device of claim 2, wherein the flexible carrier further comprises a second conductive portion disposed over the non-conductive portion and having a second sensing area for collecting a biological signal.

6. The electronic device of claim 5, wherein the flexible carrier further comprises a second connector at least partially exposed from a surface of the second conductive portion recessed from the second sensing area of the second conductive portion.

7. The electronic device of claim 6, further comprising:
a second flexible connection element having an end connecting to the second connector and an opposite end connecting to the electronic component, wherein the first flexible connection element and the second flexible connection element are arranged along the deformation direction of the electronic device sequentially.

8. The electronic device of claim 1, wherein a length the first flexible connection element is greater than a distance between two opposite ends of the first flexible connection element.

9. The electronic device of claim 2, further comprising:
a passive component disposed over the non-conductive portion; and
an antenna element disposed over the non-conductive portion,
wherein the first conductive portion, the electronic component, the passive component, and the antenna element are arranged along the deformation direction of the electronic device sequentially.

10. The electronic device of claim 10, wherein the first conductive portion, the electronic component, the passive component, and the antenna element are arranged along a signal transmission path of the electronic device sequentially.

11. An electronic device, comprising:
a flexible carrier having a first surface, a second surface opposite to the first surface, and a lateral surface extending between the first surface and the second surface;
an electronic component disposed over the second surface of the flexible carrier; and
a first sensing element disposed over the lateral surface of the flexible carrier, wherein the first sensing element includes a first conductive base and a first connector at least partially embedded in the first conductive base, and
wherein the first conductive base has a first sensing area for collecting a biological signal and substantially coplanar with the first surface of the flexible carrier.

12. The electronic device of claim 11, wherein the biological signal is transmitted to the electronic component without passing through the flexible carrier.

13. The electronic device of claim 11, wherein a part of the first connector is exposed from a surface of the first conductive base opposite to the first sensing area.

14. The electronic device of claim 13, further comprising:
an encapsulant covering the part of the first connector and the electronic component.

15. The electronic device of claim 14, wherein the first sensing area is substantially coplanar with a first surface of the encapsulant.

16. The electronic device of claim 15, further comprising:
a second sensing element disposed over the second surface of the flexible carrier,
wherein the second sensing element includes a second conductive base and a second connector at least partially embedded in the second conductive base, and
wherein the second conductive base has a second sensing area for collecting a biological signal and substantially coplanar with a second surface of the encapsulant.

17. The electronic device of claim 16, wherein the encapsulant includes a recessed portion recessed from the second surface of the encapsulant.

18. An interconnection structure, comprising:
a base having a recessed portion;
a first connector at least partially embedded in the base;
a second connector at least partially embedded in the base; and
a flexible connection element connecting the first connector and the second connector and is configured to extend along a deformation direction of the interconnection structure,
wherein the recessed portion is disposed between the first connector and the second connector and is configured to provide adjustment to a relative position between the first connector and the second connector.

19. The interconnection structure of claim 18, wherein the flexible connection element is exposed from the base.

20. The interconnection structure of claim 18, wherein the base is configured to collect a biological signal.
